(19) United States
(12) Patent Application Publication
(10) Pub. No.: US 2020/0139191 A1
(43) Pub. Date: May 7, 2020

(54) METHOD AND SYSTEM FOR STIMULATING AN INDIVIDUAL TO PROMOTE USE OF A PARETIC MEMBER INSTEAD OF A HEALTHY MEMBER

(71) Applicants: UNIVERSITE D'ANGERS, ANGERS Cedex (FR); CENTRE HOSPITALIER UNIVERSITAIRE D'ANGERS, ANGERS cedex 09 (FR)

(72) Inventor: Mickaël DINOMAIS, Feneu (FR)

(73) Assignees: UNIVERSITE D'ANGERS, ANGERS Cedex (FR); CENTRE HOSPITALIER UNIVERSITAIRE D'ANGERS, ANGERS cedex 09 (FR)

(21) Appl. No.: 16/615,259

(22) PCT Filed: May 22, 2018
(86) PCT No.: PCT/EP2018/063350

§ 371 Date: Dec. 16, 2019

(30) Foreign Application Priority Data
May 24, 2017 (FR) 1754623

(51) Int. Cl. A63B 23/16; A61B 5/11

(52) U.S. Cl. CPC A63B 23/16; A61B 5/1124

(57) ABSTRACT

A method and system for stimulating an individual in order to favour use of a paretic member with respect to a healthy member that includes an element placed on the paretic member of the individual and an object placed in the vicinity of the individual. The object includes circuitry causing the device to evaluate the distance separating the element placed on the paretic member and the object, activate a scenario for generating at least one audible and/or visual and/or somaesthetic signal if the distance separating the element placed on the paretic member and the object is less than a given distance, storing and updating information whenever the distance between the element placed on the paretic member and the object is less than the given distance.

The present invention relates to a method and system for stimulating an individual in order to promote use of a paretic member with respect to a healthy member.

Dealing with motor disorders in the case of cerebral palsy is currently a physiotherapeutic challenge.

Cerebral plasticity is a continuous process allowing medium- and long-term modifications to the synaptic organisation for better efficacy of the neural networks. Neuroplasticity is a mechanism that enables the brain to adapt its organisation to various levels according to external events in order to be as efficient as possible. Neuroplasticity underlies learning and memory processes but also cerebral reorganisation processes following a lesion to the encephalon.

In the case of unilateral cerebral palsy, establishment of immobilisation of a healthy member, and therefore over-use of the paretic member by the individual, may increase the volume of grey matter in the sensorimotor cortex of the injured hemisphere. Cerebral palsy is currently the most common cause of motor deficiency in children. Cerebral palsy is a non-developing motor disorder following a cerebral lesion occurring on the developing brain. These early cerebral lesions, occurring in the foetal life or during the neonatal period, may impair the establishment and functioning of the sensorimotor system.

Cerebral palsy is diagnosed at variable ages depending on the seriousness of the affliction. A delay in motor development may be observed by the parents as from the first months of life: absence of holding of the head at three months, absence of sitting up at three months, preferential use of only one hand.

It is desirable to establish motor physiotherapy as early as possible.

The objective of early physiotherapy is to prevent the secondary appearance of complications related to the presence of deficits, to improve the function or functions in deficit, to best promote the activities of the child and thus to allow the best possible social participation of the child.

Early physiotherapy is often effected by blocking the healthy member in the context of consultations and/or physiotherapy in a specialist medical environment. Such interventions may give rise to frustration in the individual.

The aim of the present invention is to solve the drawbacks of the prior art by proposing a method and system for stimulating an individual in order to promote use of a paretic member with respect to a healthy member on a daily basis without having to block the healthy member, without technical assistance, and in the environment of the individual so as to promote the acceptance, observance and compliance of the individual.

To this end, according to a first aspect, the invention proposes a system for stimulating an individual in order to promote use of a paretic member with respect to a healthy member, characterised in that the system comprises:
an element placed on the paretic member of the individual,
an object, placed in the vicinity of the individual, comprising:
means for evaluating the distance separating the element placed on the paretic member and the object,
means for activating a scenario for generating at least one audible and/or visual and/or somaesthetic signal if the distance separating the element placed on the paretic member and the object is less than a given distance,
means for storing and updating information whenever the distance between the element placed on the paretic member and the object is less than the given distance.

The invention also relates to a method for stimulating an individual in order to promote use of a paretic member with respect to a healthy member, characterised in that an element is placed on the paretic member of the individual and an object is placed in the vicinity of the individual, and in that the method comprises the steps, executed by the object, of:
evaluating the distance separating the element placed on the paretic member and the object,
activating a scenario for generating at least one audible and/or visual and/or somaesthetic signal if the distance separating the element placed on the paretic member and the object is less than a given distance,
storing and updating information whenever the distance between the element placed on the paretic member and the object is less than the given distance.

Thus the present invention promotes use of a paretic member with respect to a healthy member on a daily basis without having to block the healthy member, without any technical assistance and in the environment of the individual so as to promote the acceptance, observance and compliance of the individual.

By storing the information, the practitioner, when consulting this information, can thus make a connection between any progress of the individual and the use of the paretic member.

The present invention, by activating a scenario for generating at least one audible and/or visual and/or somaesthetic signal if the distance separating the element placed on the paretic member and the object is less than a given distance and not when the healthy member is close to the object, stimulates the use of the paretic member by generating a reward for the individual.

According to a particular embodiment of the invention, the updated information is the number of times when the distance between the element placed on the paretic member and the object is less than the given distance, and the information stored is the given distance and/or the scenario and/or a timestamp.

Thus the practitioner, when consulting this information, can make a connection between any progress of the individual and the use of the paretic member.

According to a particular embodiment of the invention, the object further comprises means for modifying the given distance if the number of times when the distance between the element placed on the paretic member and the object is less than the given distance is higher than a predetermined quantity.

Thus the present invention promotes motor learning. The given distance is for example large initially in order to encourage the individual to exercise, and then is reduced as the learning continues.

According to a particular embodiment of the invention, the object further comprises means for modifying the scenario if the given distance is modified.

Thus the present invention stimulates the individual by changing the scenarios over the course of time.

According to a particular embodiment of the invention, the object further comprises means for activating the scenario if a movement of the object is detected and means for modifying the scenario if a predetermined number of movements of the object are detected.

Thus the present invention stimulates the individual by changing the scenarios over time depending on the complexity of the conditions to be fulfilled in order to activate or not the reward.

According to a particular embodiment of the invention, the individual has an age of less than three years.

Thus the present invention is suited to early physiotherapy in a child.

According to a particular embodiment of the invention, the element is included in an orthosis and the object is a soft toy.

Thus the present invention is suited to early physiotherapy in a child.

According to a particular embodiment of the invention, the element is included in an orthosis and the object is a doll.

Thus the present invention is suited to physiotherapy in a child.

According to a particular embodiment of the invention, the element is included in an orthosis and the object is a small-scale vehicle model.

Thus the present invention is suited to physiotherapy in a child.

According to a particular embodiment of the invention, the element is included in an orthosis and the object is a ball.

Thus the present invention is suited to motor physiotherapy for hemiplegia.

According to a particular embodiment of the invention, the element is a permanent magnet.

Thus the manufacturing cost of the system is low.

According to a particular embodiment of the invention, the object further comprises communication means for transferring stored and updated information to a remote device.

The invention also relates to the computer programs stored on an information carrier, said programs comprising instructions for implementing the methods previously described, when they are loaded into and executed by a computer system.

The features of the invention mentioned above, as well as others, will emerge more clearly from a reading of the following description of an example embodiment, said description being given in relation to the accompanying drawings, among which:

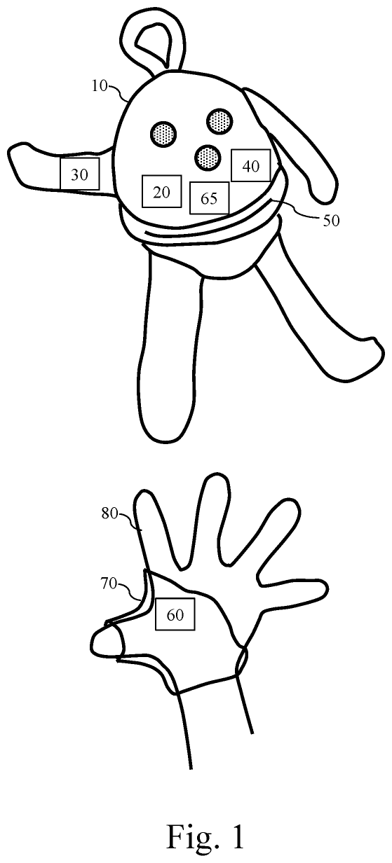

FIG. 1 depicts a system for stimulating an individual for favouring use of a paretic member with respect to a healthy member according to the present invention;

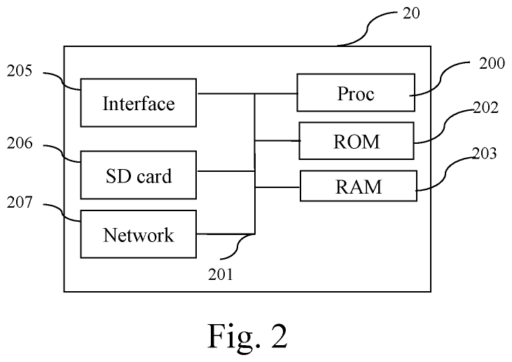

FIG. 2 depicts an example of electronic architecture of the object according to the present invention;

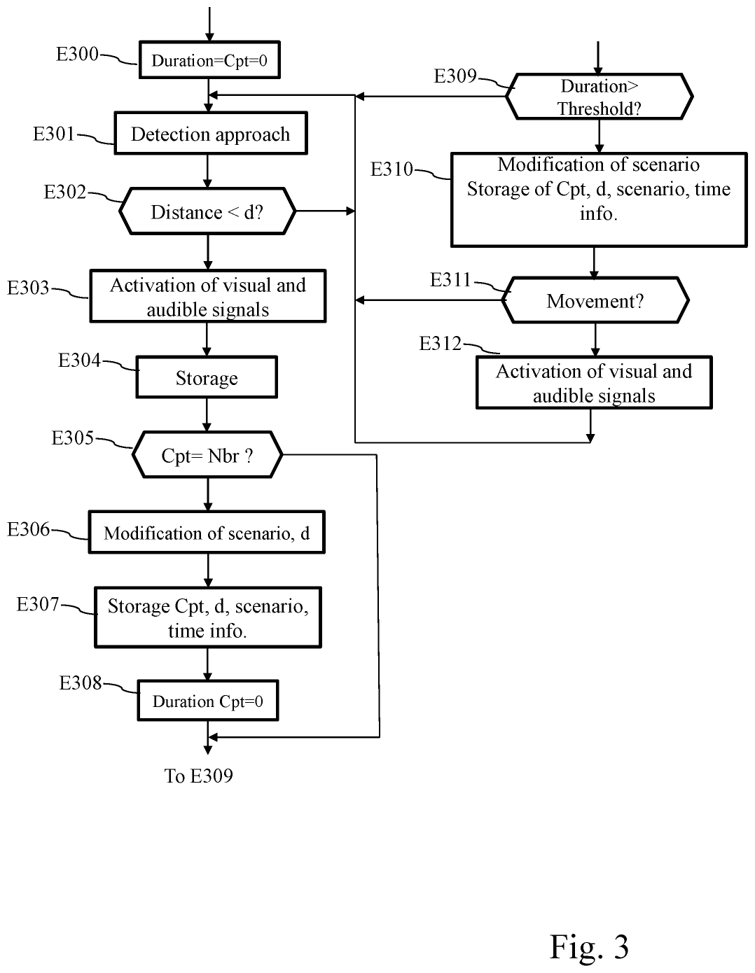

FIG. 3 depicts an algorithm for stimulating an individual in order to favour use of a paretic member with respect to a healthy member according to the present invention.

FIG. 1 depicts a system for stimulating an individual for favouring use of a paretic member with respect to a healthy member according to the present invention.

The system comprises an object 10 and an element 60 placed on a paretic member 80.

In the example in FIG. 1, the paretic member is a hand 80. Naturally the present invention finds its application with other members.

In the example in FIG. 1, the element 60 is placed in an orthosis 70. The orthosis is for example used when the paretic member has a clasped thumb. This is because the majority of children having cerebral palsy have a clasped thumb, appearing very early because of the spasticity of the extrinsic muscles or more rarely the intrinsic muscles of the hand.

Naturally the element 60 may be placed on a bracelet or a ring.

The element 60 is for example a permanent magnet.

The object 10 is, in the example in FIG. 1, a soft toy. The object has a form suited to the age of the individual. When the individual is a baby, the object is a soft toy. Naturally the object may take many forms such as for example a doll, a small-scale vehicle model or a ball.

The object 10 comprises a proximity sensor 30 that enables the distance between the object 10 and the element 60 to be determined. The proximity sensor is for example a magnetic detector when the element is a magnet.

The object 10 comprises an electronic card 20 that will be described in more detail with reference to FIG. 2.

The object 10 comprises a movement detector 60 such as for example an accelerometer and/or a gyroscope.

The object 10 comprises means for emitting audible signals 40 and means for emitting light signals 50, such as for example an array of light emitting diodes.

The object also comprises means, not shown in FIG. 1, for generating a somaesthetic signal such as the generation of vibrations or the generation of moderate heat.

FIG. 2 depicts an example of electronic architecture of the object according to the present invention.

The object 10, and more precisely the electronic card 20 of the object, comprises:
a processor, microprocessor or microcontroller 200;
a volatile memory 203;
a non-volatile memory 202;
an input-output interface 205;
a removable storage medium 206 such as for example an SD card;
a network interface 207 of the cabled or Bluetooth type makes it possible to communicate with a remote device;
a communication bus connecting the processor 200 to the ROM memory 203, to the RAM memory 203, to the input/output interface 205, to the removable storage medium 206 and to the network interface 207.

The processor 200 is capable of executing instructions loaded into the volatile memory 203 from the non-volatile memory 202, from an external memory (not shown), from a storage medium such as an SD card or the like, or from a communication network. When the object 10 is powered up, the processor 200 is capable of reading instructions from the volatile memory 203 and executing them. These instructions form a computer program that causes the implementation, by the processor 200, of all or part of the method described in relation to FIG. 3.

All or part of the method described in relation to FIG. 3 can be implemented in software form by the execution of a set of instructions by a programmable machine such as a DSP (digital signal processor) or a microcontroller, or be implemented in hardware form by a machine or a dedicated component, such as an FPGA (field-programmable gate array) or an ASIC (application-specific integrated circuit).

FIG. 3 depicts an algorithm for stimulating an individual in order to promote use of a paretic member with respect to a healthy member according to the present invention.

The present algorithm is described in an example in which it is executed by the processor 200.

At step E300, the processor 200 initialises to zero a variable denoted Duration and a variable Cpt. The variable Duration is a variable representing the value of a clock that is used for changing the scenario periodically. The variable Cpt is a counter that counts the number of times that the distance separating the element placed on the paretic member and the object is less than a given distance.

At step E301, the processor 200 obtains, by means of the input-output interface 205, the output signal from the proximity sensor 30.

At step E302, the processor 200, from the output signal of the proximity sensor 30, determines the distance separating the object 10, or more precisely the proximity sensor 30, from the element 60. The processor 200 checks whether the distance separating the element placed on the paretic member and the object is less than a given distance, for example 20 cm.

If the distance separating the element placed on the paretic member 80 and the object 10 is less than the given distance, the processor 200 passes to step E303.

If not, the processor 200 returns to step E301.

At step E303, the processor 200 triggers a scenario for generating at least one audible and/or visual and/or somaesthetic signal.

At step E304, the processor 200 stores and updates information.

The updated information is the number of times Cpt when the distance between the element placed on the paretic member and the object is less than the given distance, and the information stored is the given distance and/or the scenario and/or a timestamp.

At step E305, the processor 200 checks whether the variable Cpt is equal to a predetermined value Nbr.

For example Nbr is equal to 20.

If the variable Cpt is equal to a predetermined quantity Nbr, the processor 200 passes to step E306. If not, the processor 200 passes to step E309.

At step E306, the processor 200 modifies the scenario.

A scenario comprises a sequence of sounds and a sequence of visual animations such as a blinking of the light emitting diodes at a variable rhythm, or variations in the colours of the array of light emitting diodes.

At step E307, the processor 200 stores information.

The information stored is the given distance and/or the scenario and/or a timestamp.

At step E308, the processor 200 sets the variable Cpt to zero.

At step E309, the processor 200 checks whether the variable Duration is higher than a threshold, for example equal to 24 hours.

If the variable Duration is higher than the threshold, the processor 200 passes to step E310 and sets the variable Duration to zero. If not, the processor 200 returns to step E301.

At step E310, the processor 200 modifies the scenario, and stores and updates information.

The information updated is the number of times Cpt when the distance between the element placed on the paretic member and the object is less than the given distance, and the information stored is the given distance and/or the scenario and/or a timestamp.

At step E311, the processor 200 checks whether a movement of the object is detected by the movement detector 60.

If a movement of the object is detected, the processor 200 passes to step E312. If not, the processor 200 returns to step E301.

At step E312, the processor 200 triggers a scenario for generating at least one audible and/or visual and/or somaesthetic signal.

The processor 200 next returns to step E301.

Naturally the present invention is in no way limited to the embodiments described here, but quite the contrary encompasses any variant within the capability of a person skilled in the art.

In particular, the present invention, in an example in which the processor 200 modifies the scenario, stores and updates information according to the number of times when the distance separating the element placed on the paretic member and the object is less than a given distance.

The modification of the scenario and the storage and updating of the information can also be carried out according to the number of movements of the object detected.

1. A system for stimulating an individual in order to promote use of a paretic member with respect to a healthy member, wherein the system comprises:
an element placed on the paretic member of the individual,
an object, placed in the vicinity of the individual, the object comprising:
evaluating the distance separating the element placed on the paretic member and the object,
activating a scenario for generating at least one audible and/or visual and/or somaesthetic signal if the distance separating the element placed on the paretic member and the object is less than a given distance,
storing and updating information whenever the distance between the element placed on the paretic member and the object is less than the given distance.

2. The system according to claim 1, wherein the updated information is the number of times when the distance between the element placed on the paretic member and the object is less than the given distance, and the information stored is the given distance and/or the scenario and/or a timestamp.

3. The system according to claim 2, wherein the object further comprises means for modifying the given distance if the number of times when the distance between the element placed on the paretic member and the object is less than the given distance is higher than a predetermined quantity.

4. The system according to claim 2, wherein the object further comprises means for modifying the scenario if the given distance is modified.

5. The system according to claim 2, wherein the object further comprises means for activating the scenario if a movement of the object is detected and means for modifying the scenario if a predetermined number of movements of the object are detected.

6. The system according to claim 5, wherein the element is included in an orthosis and the object is a soft toy or a doll or a ball or a small-scale vehicle model.

7. The system according to claim 6, wherein the element is a permanent magnet.

8. The system according to claim 1, wherein the object further comprises communication means for transferring stored and updated information to a remote device.
